(19) United States
(12) Patent Application Publication
(10) Pub. No.: US 2004/0192674 A1
(43) Pub. Date: Sep. 30, 2004

(54) Cathepsin L inhibitors

(76) Inventor: Robert W. Marquis, Collegeville, PA (US)

Correspondence Address: GLAXOSMITHKLINE, Corporate Intellectual Property - UW2220, P.O. Box 1539, King of Prussia, PA, 19406-0939, US

(21) Appl. No.: 10/772,817

(22) Filed: Feb. 5, 2004

Related U.S. Application Data
(60) Provisional application No. 60/447,558, filed on Feb. 14, 2003.

(51) Int. Cl. A61K 31/55

(52) U.S. Cl. 514/217.04; 514/217.05; 514/217.07; 514/217.11

(57) ABSTRACT

The present invention provides methods which uses certain 4-amino-azepan-3-ones to inhibit cathepsin L. Consequently they are useful for preventing or treating diseases in which cathepsin L is implicated, such as rheumatoid arthritis or inhibition of positive selection of CD4⁺T-cells by cortical thymic epithelial cells.

### FIELD OF THE INVENTION

This invention relates in general to the use of 4-amino-azepan-3-one protease inhibitors, particularly such inhibitors of cathepsin L, in the treatment or prevention of diseases in which cathepsin L is implicated. Those diseases include for example autoimmune diseases, injury arising from the formation of atherosclerotic lesions and complications arising therefrom, diseases requiring inhibition for therapy of a class II MHC-restricted immune response, inhibition of an asthmatic response, inhibition of an allergic response, inhibition of immune response against a transplanted organ or tissue, or inhibition of elastase activity in atheroma.

### BACKGROUND OF THE INVENTION

Cathepsins are a family of enzymes that are part of the papain superfamily of cysteine proteases. Cathepsins B, H, L, N and S have been described in the literature.

Cathepsins function in the normal physiological process of protein degradation in animals, including humans, e.g., in the degradation of connective tissue. However, elevated levels of these enzymes in the body can result in pathological conditions leading to disease. Thus, cathepsins have been implicated as causative agents in various disease states, including but not limited to, infections by pneumocystis carinii, trypsanoma cruzi, trypsanoma brucei brucei, and Crithidia fusiculata; as well as in schistosomiasis, malaria, tumor metastasis, metachromatic leukodystrophy, muscular dystrophy, amytrophy, and the like. See International Publication Number WO 94/04172, published on Mar. 3, 1994, and references cited therein. See also European Patent Application EP 0 603 873 A1, and references cited therein. Two bacterial cysteine proteases from P. gingivallis, called gingipains, have been implicated in the pathogenesis of gingivitis. Potempa, J., et al. (1994) Perspectives in Drug Discovery and Design, 2, 445-458.

Pathological levels of cathepsin L have been implicated in several disease states. Thus, selective inhibition of cathepsin L may provide an effective treatment for diseases requiring, for therapy or prevention: inhibition of rheumatoid arthritis (see Iwata et. al. Arthritis and Rheumatism 1997, 40, 499), inhibition of cancer metastasis (see K. Ishidoh and E. Kominami Biol. Chem. 1998, 379, 131, inhibition of positive selection of CD4⁺ T-cells by cortical thymic epithelial cells (Nakagawa Science 1998, 270, 450).

Several cysteine protease inhibitors are known. Palmer, (1995) J. Med. Chem., 38, 3193, disclose certain vinyl sulfones which irreversibly inhibit cysteine proteases, such as the cathepsins B, L, S, O2 and cruzain. Other classes of compounds, such as aldehydes, nitriles, α-ketocarbonyl compounds, halomethyl ketones, diazomethyl ketones, (acyloxy)methyl ketones, ketomethylsulfonium salts and epoxy succinyl compounds have also been reported to inhibit cysteine proteases. See Palmer, id, and references cited therein.

U.S. Pat. No. 4,518,528 discloses peptidyl fluoromethyl ketones as irreversible inhibitors of cysteine protease. Published International Patent Application No. WO 94/04172, and European Patent Application Nos. EP 0 525 420 A1, EP 0 603 873 A1, and EP 0 611 756 A2 describe alkoxymethyl and mercaptomethyl ketones which inhibit the cysteine proteases cathepsins B, H and L. International Patent Application No. PCT/US94/08868 and European Patent Application No. EP 0 623 592 A1 describe alkoxymethyl and mercaptomethyl ketones which inhibit the cysteine protease IL-1βconvertase. Alkoxymethyl and mercaptomethyl ketones have also been described as inhibitors of the serine protease kininogenase (International Patent Application No. PCT/GB91/01479).

Azapeptides, which are designed to deliver the azamino acid to the active site of serine proteases, and which possess a good leaving group, are disclosed by Elmore et al., Biochem. J., 1968, 107, 103, Garker et al., Biochem. J., 1974, 139, 555, Gray et al., Tetrahedron, 1977, 33, 837, Gupton et al., J. Biol. Chem., 1984, 259, 4279, Powers et al., J. Biol. Chem., 1984, 259, 4288, and are known to inhibit serine proteases. In addition, J. Med. Chem., 1992, 35, 4279, discloses certain azapeptide esters as cysteine protease inhibitors.

Antipain and leupeptin are described as reversible inhibitors of cysteine protease in McConnell et al., J. Med. Chem., 33, 86; and also have been disclosed as inhibitors of serine protease in Umezawa et al., 45 Meth. Enzymol. 678. E64 and its synthetic analogs are also well-known cysteine protease inhibitors (Barrett, Biochem. J., 201, 189, and Grinde, Biochem. Biophys. Acta, 701, 328).

1,3-diamido-propanones have been described as analgesic agents in U.S. Pat. Nos. 4,749,792 and 4,638,010.

A variety of cysteine and serine protease inhibitors, especially of cathepsin K, have been disclosed in International Publication Number WO 97/16433, published on May 9, 1997.

Thus, a structurally diverse variety of protease inhibitors have been identified. However, these known inhibitors are not considered suitable for use as therapeutic agents in animals, especially humans, because they suffer from various shortcomings. These shortcomings include lack of selectivity, cytotoxicity, poor solubility, and overly rapid plasma clearance. A need therefore exists for methods of treating diseases caused by pathological levels of proteases, particularly cysteine proteases, more particularly cathepsins, most particularly cathepsin L, and for novel inhibitor compounds useful in such methods.

We have now discovered that the present 4-amino-azepan-3-one compounds inhibit cathepsin L, and are useful in the treatment of diseases in which cathepsin L is implicated.

### SUMMARY OF THE INVENTION

An object of the present invention is to provide a method for preventing or treating diseases which may be therapeutically modified by altering the activity of cathepsin L, the method comprising administering a 4-amino-azepan-3-one of Formula I which are protease inhibitors of cathepsin L.

In a particular aspect, the methods of this invention are especially useful for treatment or prevention of diseases requiring, for therapy, inhibition rheumatoid arthritis, cancer metastasis, or inhibition of positive selection of CD4⁺ T-cells by cortical thymic epithelial cells.

### DETAILED DESCRIPTION OF THE INVENTION

The present invention provides a method of inhibiting cathepsin L comprising administering to an animal, particularly a mammal, most particularly a human being in need thereof, an effective amount of a compound of Formula I:

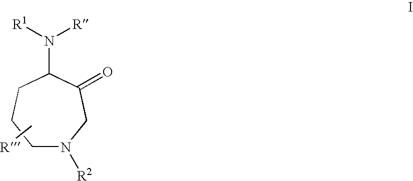

wherein:

R¹ is

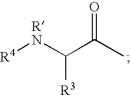

R² is H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹(R¹¹)NSO₂—

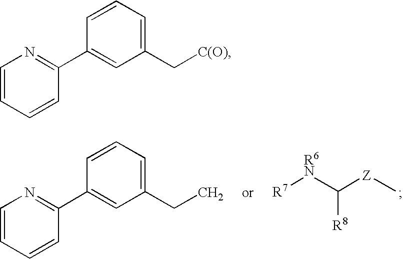

R³ is H, C₁₋₆alkyl, C₂₋₆alkenyl, C₂₋₆alkynyl, HetC₀₋₆alkyl or ArC₀₋₆alkyl;

R³ and R′ may be connected to form a pyrrolidine, piperidine or morpholine ring;

R⁴ is R⁵C(O)—;

R⁵ is quinolin-6-yl;

R⁶ is H, C₁₋₆alkyl, Ar—C₀₋₆alkyl, or Het-C₀₋₆alkyl;

R⁷ is H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰C(O)—, R¹⁰R¹⁴NC(O)—, or R¹⁰R¹⁴NC(S)—;

R⁸ is H, C₁₋₆alkyl, C₂₋₆alkenyl, C₂₋₆alkynyl, HetC₀₋₆alkyl or ArC₀₋₆alkyl;

R⁹ is C₁₋₆alkyl, C₃₋₆cycloalkyl-C₀₋₆alkyl, Ar—C₀₋₆alkyl or Het-C₀₋₆alkyl;

R¹⁰ is C₁₋₆alkyl, C₃₋₆cycloalkyl-C₀₋₆alkyl, Ar—C₀₋₆alkyl or Het-C₀₋₆alkyl;

R¹¹ is H, C₁₋₆alkyl, Ar—C₀₋₆alkyl, or Het-C₀₋₆alkyl;

R¹² is H, C₁₋₆alkyl, Ar—C₀₋₆alkyl, or Het-C₀₋₆alkyl;

R¹³ is H, C₁₋₆alkyl, Ar—C₀₋₆alkyl, or Het-C₀₋₆alkyl;

R¹⁴ is H, C₁₋₆alkyl, Ar—C₀₋₆alkyl, or Het-C₀₋₆alkyl;

R′ is H, C₁₋₆alkyl, Ar—C₀₋₆alkyl, or Het-C₀₋₆alkyl;

R″ is H, C₁₋₆alkyl, Ar—C₀₋₆alkyl, or Het-C₀₋₆alkyl;

R′″ is H, C₁₋₆alkyl, C₃₋₆cycloalkyl-C₀₋₆alkyl, Ar—C₀₋₆alkyl, or Het-C₀₋₆alkyl;

X is CH₂, S, or O;

Z is C(O) or CH₂;

and pharmaceutically acceptable salts, hydrates and solvates thereof.

In compounds of Formula I, preferably:

R³ is H, C₁₋₆alkyl, C₂₋₆alkenyl, C₂₋₆alkynyl, Het-C₀₋₆alkyl and Ar—C₀₋₆alkyl, preferably C₁₋₆alkyl and Ar—C₀₋₆alkyl, most preferably isobutyl, napthalen-2-ylmethyl, benzyl, or benzyloxymethyl;

isoquinolinyl, especially isoquinolin-1-yl

benzofuranyl, especially benzofuran-2-yl.

R′ is H, C₁₋₆alkyl, Ar—C₀₋₆alkyl, or Het-C₀₋₆alkyl, preferably H.

R″ is H, C₁₋₆alkyl, Ar—C₀₋₆alkyl, or Het-C₀₋₆alkyl, preferably H.

In compounds of Formula I R² is preferably R⁹SO₂.

R⁹ is preferably Het-C₀₋₆alkyl, and more preferably pyridinyl and 1-oxy-pyridinyl. When R² is R⁹SO₂, R⁹ is even more preferably pyridin-2-yl or 1-oxy-pyridin-2-yl. Most preferably, R⁹ is 1-oxy-pyridin-2-yl.

Most preferred are compounds of Formula I wherein:

R¹ is

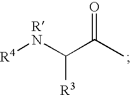

R² is R⁹SO₂;

R³ is isobutyl, napthalen-2-ylmethyl, benzyl, or benzyloxymethyl;

R⁴ is R⁵C(O);

R⁵ is quinolin-6-yl;

R⁹ is pyridin-2-yl or 1-oxy-pyridin-2-yl, preferably 1-oxy-pyridin-2-yl.

R′ is H

R″ is H; and

R′″ is H;

The following compounds of Formula I are particularly preferred:

quinoline-6-carboxylic acid {(S)-naphthylen-2-yl-1-[(S)-oxo-1-(pyridine-2-sulfonyl)-azepan-4-yl carbamoyl]-ethyl}-amide (formula A);

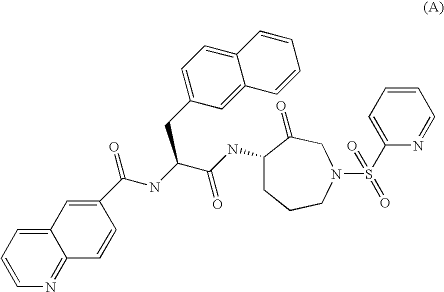

and

quinoline-6-carboxylic acid {(S)-1-[(S)-3-oxo-1-(pyridine-2-sulfonyl)azepan-4-yl carbamoyl]-2-phenyl-ethyl}-amide (formula B).

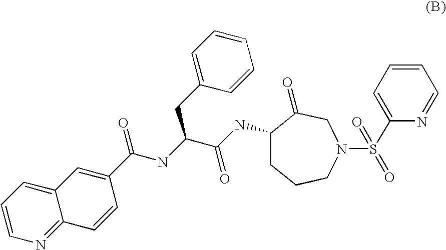

Specific representative compounds used in the present invention are set forth below.

Compared to the corresponding 5 and 6 membered ring compounds, the 7 membered ring compounds used in the present invention are configurationally more stable at the carbon center alpha to the ketone.

Definitions

The compounds used in the present invention include all hydrates, solvates, complexes and prodrugs. Prodrugs are any covalently bonded compounds, which release the active parent drug according to Formula I in vivo. If a chiral center or another form of an isomeric center is present in a compound used in the present invention, all forms of such isomer or isomers, including enantiomers and diastereomers, are intended to be covered herein. Compounds used in the present methods containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any subformula thereof is independent of its meaning, or any other substituent's meaning, at any other occurrence, unless specified otherwise.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

“Proteases” are enzymes that catalyze the cleavage of amide bonds of peptides and proteins by nucleophilic substitution at the amide bond, ultimately resulting in hydrolysis. Such proteases include: cysteine proteases, serine proteases, aspartic proteases, and metalloproteases. The compounds of the present invention are capable of binding more strongly to the enzyme than the substrate and in general are not subject to cleavage after enzyme catalyzed attack by the nucleophile. They therefore competitively prevent proteases from recognizing and hydrolyzing natural substrates and thereby act as inhibitors.

The term “amino acid” as used herein refers to the D- or L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

“C₁₋₆alkyl” as applied herein is meant to include substituted and unsubstituted methyl, ethyl, n-propyl, isopropyl, n-butyl, isobutyl and t-butyl, pentyl, n-pentyl, isopentyl, neopentyl and hexyl and the simple aliphatic isomers thereof. C₁₋₆alkyl may be optionally substituted by a moiety selected from the group consisting of: OR¹², C(O)R¹², SR¹², S(O)R¹², NR¹²₂, R¹²NC(O)OR⁵, CO₂R¹², CO₂NR¹²₂, N(C═NH)NH₂, Het, C₃₋₆cycloalkyl, and Ar; where R⁵ is selected from the group consisting of: H, C₁₋₆alkyl, C₂₋₆alkenyl, C₂₋₆alkynyl, C₃₋₆cycloalkyl-C₀₋₆alkyl, Ar—C₀₋₆alkyl and Het-C₀₋₆alkyl; and R¹² is selected from the group consisting of: H, C₁₋₆alkyl, Ar—C₀₋₆alkyl, and Het-Co ₆alkyl;

“C₃₋₆cycloalkyl” as applied herein is meant to include substituted and unsubstituted cyclopropane, cyclobutane, cyclopentane and cyclohexane.

“C₂₋₆ alkenyl” as applied herein means an alkyl group of 2 to 6 carbons wherein a carbon-carbon single bond is replaced by a carbon-carbon double bond. C₂₋₆alkenyl includes ethylene, 1-propene, 2-propene, 1-butene, 2-butene, isobutene and the several isomeric pentenes and hexenes. Both cis and trans isomers are included.

“C₂₋₆alkynyl” means an alkyl group of 2 to 6 carbons wherein one carbon-carbon single bond is replaced by a carbon-carbon triple bond. C₂₋₆ alkynyl includes acetylene, 1-propyne, 2-propyne, 1-butyne, 2-butyne, 3-butyne and the simple isomers of pentyne and hexyne.

“Halogen” means F, Cl, Br, and I.

“Ar” or “aryl” means phenyl or naphthyl, optionally substituted by one or more of Ph—C₀₋₆alkyl; Het-C₀₋₆alkyl; C₁₋₆alkoxy; Ph—C₀₋₆alkoxy; Het-C₀₋₆alkoxy; OH, (CH₂)₁₋₆NR¹⁵R¹⁶; O(CH₂)₁₋₆NR¹⁵R¹⁶; C₁₋₆alkyl, OR¹⁷, N(R¹⁷)₂, SR¹⁷, CF₃, NO₂, CN, CO₂R¹⁷, CON(R¹⁷), F, Cl, Br or 1; where R¹⁵ and R¹⁶ are H, C₁₋₆alkyl, Ph—C₀₋₆alkyl, naphthyl-C₀₋₆alkyl or Het-C₀₋₆alkyl; and R¹⁷ is phenyl, naphthyl, or C₁₋₆alkyl.

As used herein “Het” or “heterocyclic” represents a stable 5- to 7-membered monocyclic, a stable 7- to 10-membered bicyclic, or a stable 11- to 18-membered tricyclic heterocyclic ring which is either saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure, and may optionally be substituted with one or two moieties selected from C₀₋₆Ar, C₁₋₆alkyl, OR¹⁷, N(R¹⁷)₂, SR¹⁷, CF₃, NO₂, CN, CO₂R¹⁷, CON(R¹⁷), F, Cl, Br and I, where R¹⁷ is phenyl, naphthyl, or C₁₋₆alkyl. Examples of such heterocycles include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, pyridinyl, 1-oxo-pyridinyl, pyrazinyl, oxazolidinyl, oxazolinyl, oxazolyl, isoxazolyl, morpholinyl, thiazolidinyl, thiazolinyl, thiazolyl, quinuclidinyl, indolyl, quinolinyl, quinoxalinyl, isoquinolinyl, benzimidazolyl, benzopyranyl, benzoxazolyl, furanyl, benzofuranyl, thiophenyl, benzo[b]thiophenyl, thieno[3,2-b]thiophenyl, benzo[1,3]dioxolyl, 1,8 naphthyridinyl, pyranyl, tetrahydrofuranyl, tetrahydropyranyl, thienyl, benzoxazolyl, thiamorpholinyl sulfoxide, thiamorpholinyl sulfone, and oxadiazolyl, as well as triazolyl, thiadiazolyl, oxadiazolyl, isothiazolyl, iniidazolyl, pyridazinyl, pyrimidinyl, triazinyl and tetrazinyl which are available by routine chemical synthesis and are stable. The term heteroatom as applied herein refers to oxygen, nitrogen and sulfur.

Here and throughout this application the term CO denotes the absence of the substituent group immediately following; for instance, in the moiety ArC₀₋₆alkyl, when C is 0, the substituent is Ar, e.g., phenyl. Conversely, when the moiety ArC₀₋₆alkyl is identified as a specific aromatic group, e.g., phenyl, it is understood that the value of C is 0.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

Certain reagents are abbreviated herein. m-CPBA refers to 3-chloroperoxybenzoic acid, EDC refers to N-ethyl-N′(dimethylaminopropyl)-carbodiimide, DMF refers to dimethyl formamide, DMSO refers to dimethyl sulfoxide, TEA refers to triethylamine, TFA refers to trifluoroacetic acid, and THF refers to tetrahydrofuran.

Methods of Preparation

Compounds of the general formula I may be prepared in a fashion analogous to that outlined in Schemes 1, 2 and 3. Alkylation of benzyl-N-allylcarbamate (1) with a base such as sodium hydride and 5-bromo-1-pentene provides the diene 2 (Scheme 1). Treatment of 2 with bis(tricyclohexylphosphine)benzylidine ruthenium (IV) dichloride catalyst developed by Grubbs provides the tetrahydroazepine 3. Epoxidation of 3 may be effected with an oxidizing agent common to the art such as m-CPBA to provide the epoxide 4. Nucleophilic ring opening of epoxide 4 may be effected with a reagent such as sodium azide to provide the azido alcohol 5 which may be reduced to the amino alcohol 6 under conditions common to the art such as 1,3-propanedithiol and triethylamine in methanol or triphenylphosphine in THF and water. The amine of compound 6 may be protected with di-tert-butyl dicarbonate to provide derivative 7 (Scheme 2). Removal of the benzyloxycarbonyl protecting group may be effected by treatment of 7 with hydrogen gas in the presence of a catalyst such as 10% Pd/C to provide the amine 8. Treatment of amine 8 with a sulfonyl chloride such as 2-pyridinesulfonyl chloride in the presence of a base such as triethylamine provides the sulfonamide derivative 9. Removal of the tert-butoxycarbonyl protecting group may be effected with an acid such as hydrochloric acid to provide intermediate 10. Coupling of 10 with an acid such as N-Boc-p-naphthylalanine in the presence of a coupling agent common to the art such as HBTU or polymer supported EDC provides the alcohol intermediate 11. Removal of the tert-butoxycarbonyl protecting group under acidic conditions provides 12. Coupling of 12 with an acid such as quinoline-6-carboxylic acid in the presence of a coupling agent such as HBTU or polymer supported EDC provides alcohol 13. Alcohol 13 may be oxidized with an oxidant common to the art such as pyridine sulfur trioxide complex in DMSO and triethylamine or the Dess-Martin periodinane to provide the ketone 14. The diastereomers of 14 may be separated by HPLC.

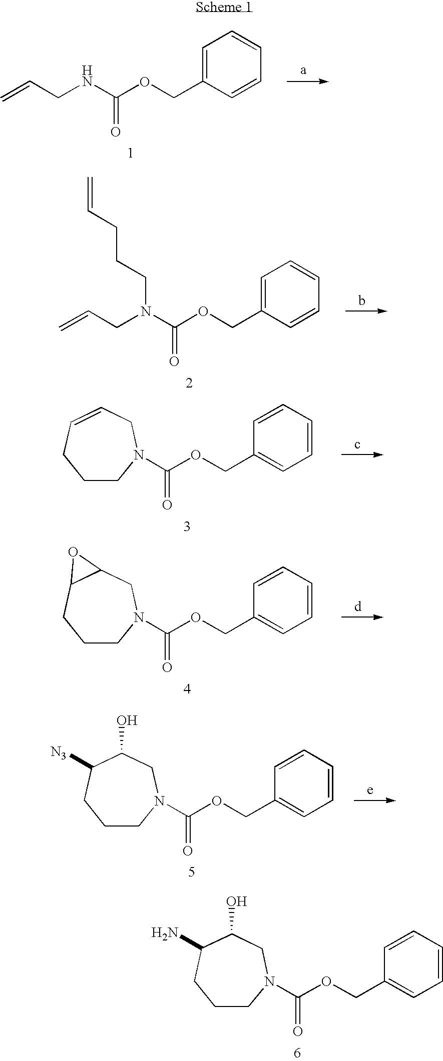

Reagents and conditions: (a) NaR, 5-bromo-1-pentene, NaH; (b) bis(tricyclohexylphosphine)benzylidine ruthenium (IV) dichloride, CH₂Cl₂, reflux; (c) m-CPBA, CH₂Cl₂; (d) NaN₃, NH₄Cl, CH₃OH, H₂O; (e) TEA, 1,3-propanedithiol, CH₃OH.

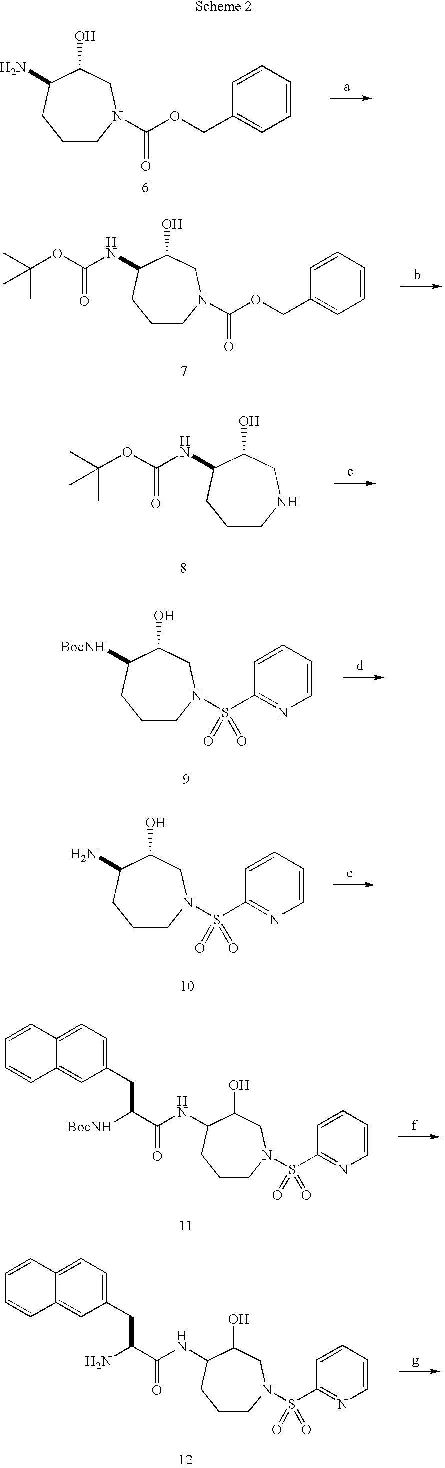

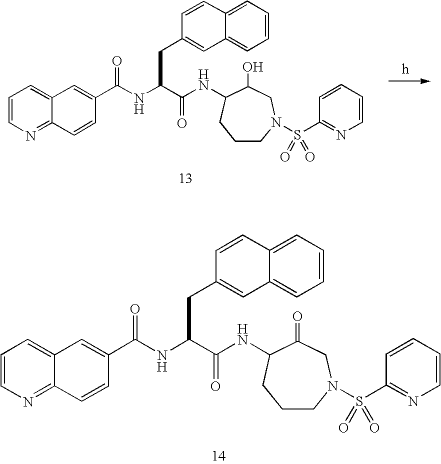

Reagents and conditions: (a) Di-tert-butyl dicarbonate, THF; (b) H₂, 10% Pd/C, EtOAc; (c) 2-pyridinesulfonyl chloride, TEA, CH₂Cl₂; (d) HCl, EtOAc; (e) N-Boc-p-naphthylalanine, P-EDC, CH₂Cl₂; (f) HCl, CH₂Cl₂; (g) quinoline-6-carboxylic acid, P-EDC, CH₂Cl₂; (h) Dess-Martin periodinane, methylene chloride.

Alternatively compounds for the general formula I may be prepared as shown in Scheme 3. Acylation of the amino alcohol 6 with an acid such as N-Boc-phenylalanine in the presence of a coupling agent such as EDC or HBTU provides the amide 15. Hydrogenolysis of the carbonylbenzyloxy protecting group employing conditions known in the art such as hydrogen gas in the presence of a catalyst such as 10% Pd/C gives the amine 16. Treatment of amine 16 with a sulfonyl chloride such as 2-pyridinesulfonyl chloride in the presence of a base

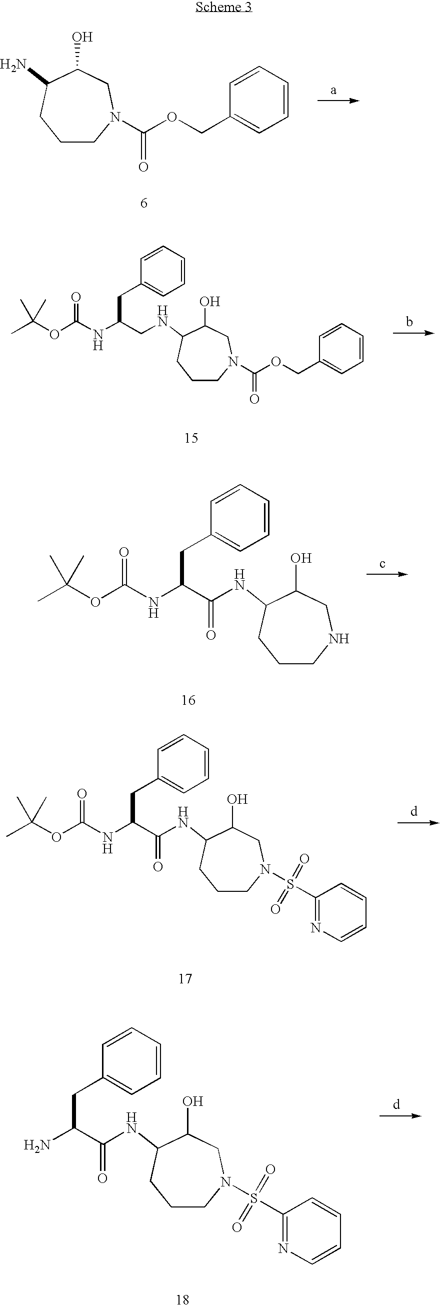

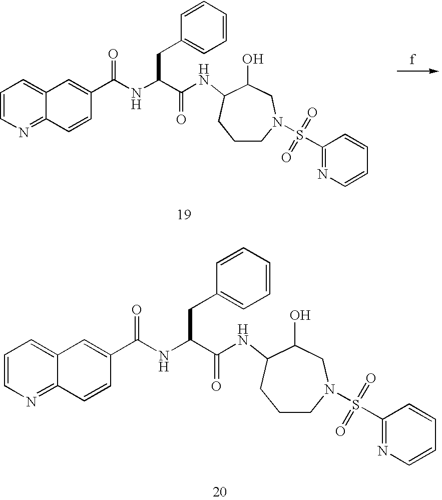

Reagents and conditions: (a) N-Boc-phenylalanine, EDC, HOBt, TEA, CH₂Cl₂; (b) H₂, 10% Pd/C, EtOAc; (c) 2-pyridinesulfonyl chloride, TEA, CH₂Cl₂; (d) HCl, methanol; (e) quinoline-6-carboxylic acid, EDC, HOBt, TEA, CH₂Cl₂; (f) pyridine sulfur trioxide complex, TEA, DMSO.

such as triethylamine provides the sulfonamide derivative 17. Removal of the tert-butoxycarbonyl protecting group may be effected with an acid such as hydrochloric acid to provide intermediate 18. Coupling of 18 with an acid such as quinoline-7-carboxylic acid in the presence of a coupling agent common to the art such as HBTU or EDC provides intermediate 19. Alcohol 19 may be oxidized with an oxidant common to the art such as pyridine sulfur trioxide complex in DMSO and triethylamine or the Dess-Martin periodinane to provide the ketone 20. The diastereomers of 20 may be separated by HPLC.

The starting materials used herein are commercially available amino acids or are prepared by routine methods well known to those of ordinary skill in the art and can be found in standard reference books, such as the COMPENDIUM OF ORGANIC SYNTHETIC METHODS, Vol. I-VI (published by Wiley-Interscience).

Coupling methods to form amide bonds herein are generally well known to the art. The methods of peptide synthesis generally set forth by Bodansky et al., THE PRACTICE OF PEPTIDE SYNTHESIS, Springer-Verlag, Berlin, 1984; E. Gross and J. Meienhofer, THE PEPTIDES, Vol. 1, 1-284 (1979); and J. M. Stewart and J. D. Young, SOLID PHASE PEPTIDE SYNTHESIS, 2d Ed., Pierce Chemical Co., Rockford, III., 1984; are generally illustrative of the technique and are incorporated herein by reference.

Synthetic methods to prepare the compounds of this invention frequently employ protective groups to mask a reactive functionality or minimize unwanted side reactions. Such protective groups are described generally in Green, T. W, PROTECTIVE GROUPS IN ORGANIC SYNTHESIS, John Wiley & Sons, New York (1981). The term “amino protecting groups” generally refers to the Boc, acetyl, benzoyl, Fmoc and Cbz groups and derivatives thereof as known to the art. Methods for protection and deprotection, and replacement of an amino protecting group with another moiety are well known.

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts. Halides, sulfate, phosphate, alkanoates (such as acetate and trifluoroacetate), benzoates, and sulfonates (such as mesylate) are examples of anions present in pharmaceutically acceptable salts.

Administering a pharmaceutical composition, which comprises a compound according to Formula, I and a pharmaceutically acceptable carrier, diluent or excipient, may practice the methods of the present invention. Accordingly, the compounds of Formula I may be used in the manufacture of a medicament. Pharmaceutical compositions of the compounds of Formula I prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of syrup, elixir, emulsion or an aqueous or non-aqueous suspension. Such a liquid formulation may be administered directly p.o. or filled into a soft gelatin capsule.

For rectal administration, the compounds of this invention may also be combined with excipients such as cocoa butter, glycerin, gelatin or polyethylene glycols and molded into a suppository.

### Utility of the Present Invention

The compounds of Formula I are useful as inhibitors of cathepsin L. The present invention provides methods of treatment of diseases caused by pathological levels of cathepsin L, which methods comprise administering to an animal, particularly a mammal, most particularly a human in need thereof a therapeutically effective amount of an inhibitor of cathepsin L, including a compound of the present invention.

The present invention particularly provides methods for treating the following diseases in which cathepsin L is implicated:

Diseases, which require for therapy: inhibition of rheumatoid arthritis, inhibition of cancer metastasis, or inhibition of positive selection of CD4⁺ T-cells by cortical thymic epithelial cells.

The present methods contemplate the use of one or more compounds of Formula 1, alone or in combination with other therapeutic agents.

For acute therapy, parenteral administration of a compound of Formula I is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit cathepsin S. The compounds are administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of Formula I may also be administered orally to the patient, in a manner such that the concentration of drug is sufficient to inhibit rheumatoid arthritis or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of Formula I are administered in accordance with the present methods.

### Biological Assays

The compounds used in the present methods may be tested in one of several biological assays to determine the concentration of compound, which is required to have a given pharmacological effect.

Determination of Cathepsin L Proteolytic Catalytic Activity

All assays for cathepsin L were carried out with human recombinant enzyme. Standard assay conditions for the determination of kinetic constants used a fluorogenic peptide substrate, typically Cbz-Phe-Arg-AMC, and were determined in 100 mM Na acetate at pH 5.5 containing 20 mM cysteine and 5 mM EDTA. Stock substrate solutions were prepared at concentrations of 10 or 20 mM in DMSO with 20 μM final substrate concentration in the assays. All assays contained 10% DMSO. All assays were conducted at ambient temperature. Product fluorescence (excitation at 360 nM; emission at 460 nM) was monitored with a Perceptive Biosystems Cytofluor II fluorescent plate reader. Product progress curves were generated over 20 to 30 minutes following formation of AMC product.

Inhibition Studies

Potential inhibitors were evaluated using the progress curve method. Assays were carried out in the presence of variable concentrations of test compound. Reactions were initiated by addition of enzyme to buffered solutions of inhibitor and substrate. Data analysis was conducted according to one of two procedures depending on the appearance of the progress curves in the presence of inhibitors. For those compounds whose progress curves were linear, apparent inhibition constants (K_{i,app}) were calculated according to equation 1 (Brandt et al., Biochemitsry, 1989, 28, 140):

v=VₘA/[Kₐ(I+I/K_{i, app})+A] (1)

where v is the velocity of the reaction with maximal velocity Vₘ, A is the concentration of substrate with Michaelis constant of Kₐ and I is the concentration of inhibitor.

For those compounds whose progress curves showed downward curvature characteristic of time-dependent inhibition, the data from individual sets was analyzed to give k_{obs} according to equation 2:

[AMC]=vₛₛt+(v₀−vₛₛ) [1−exp(−k_{obs}t)]/k_{obs} (2)

where [AMC] is the concentration of product formed over time t, v₀ is the initial reaction velocity and vₛₛ is the final steady state rate. Values for k_{obs} were then analyzed as a linear function of inhibitor concentration to generate an apparent second order rate constant (k_{obs}/inhibitor concentration or k_{obs}/[I]) describing the time-dependent inhibition. A complete discussion of this kinetic treatment has been fully described (Morrison et al., Adv. Enzymol. Relat. Areas Mol. Biol., 1988, 61, 201).

### General

Nuclear magnetic resonance spectra were recorded at either 250 or 400 MHz using, respectively, a Bruker AM 250 or Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (d) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (FTIR) spectra were recorded on a Nicolet Impact 400 D infrared spectrometer. IR and FTIR spectra were recorded in transmission mode, and band positions are reported in inverse wavenumbers (cm⁻¹). Mass spectra were taken on either VG 70 FE, PE Syx API III, or VG ZAB HF instruments, using fast atom bombardment (FAB) or electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel.

Where indicated, certain of the materials were purchased from the Aldrich Chemical Co., Milwaukee, Wis., Chemical Dynamics Corp., South Plainfield, N.J., and Advanced Chemtech, Louisville, Ky.

### Examples

In the following synthetic examples, temperature is in degrees Centigrade (° C.). Unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

### Example 1

Preparation of Quinoline-6-carboxylic acid {(S)-1-[(S)-3-oxo-1-(pyridine-2-sulfonyl)azepan-4-yl carbamoyl]-2-phenyl-ethyl}-amide

a.) Allyl-pent-4-enyl-carbamic acid benzyl ester

To a suspension of NaH (1.83 g, 76.33 mmol of 90% NaH) in DMF was added allyl-carbamic acid benzyl ester (7.3 g, 38.2 mmol) in a dropwise fashion. The mixture was stirred at room temperature for approximately 10 minutes whereupon 5-bromo-1-pentene (6.78 mL, 57.24 mmol) was added in a dropwise fashion. The reaction was heated to 40° C. for approximately 4 hours whereupon the reaction was partitioned between dichloromethane and water. The organic layer was washed with water (2×'s), brine, dried (MgSO₄), filtered and concentrated. Column chromatography of the residue (10% ethyl acetate:hexanes) provided 10.3 grams of the title compound as an oil: MS(EI) 260 (M+H⁺).

b.) 2,3,4,7-Tetrahydro-azepine-1-carboxylic acid benzyl ester

To a solution of compound of Example 1a (50 g) in dichloromethane was added bis(tricyclohexylphosphine)benzylidine ruthenium (IV) dichloride (5.0 g). The reaction was heated to reflux until complete as determined by TLC analysis. The reaction was concentrated in vacuo. Column chromatography of the residue (50% dichloromethane:hexanes) gave 35 g of the title compound: MS(EI) 232 (M+H⁺).

c.) 8-Oxa-3-aza-bicyclo[5.1.0]octane-3-carboxylic acid benzyl ester

To a solution of the compound of Example 1b (35 g, 1.5 mol) in dichloromethane was added m-CPBA (78 g, 0.45 mol). The mixture was stirred overnight at room temperature whereupon it was filtered to remove the solids. The filtrate was washed with saturated water and saturated NaHCO₃ (several times). The organic layer was dried (MgSO₄), filtered and concentrated to give 35 g of the title compound which was of sufficient purity to carry on to the next step: MS(EI) 248 (M+H⁺), 270 (M+Na⁺).

d. 4-Azido-3-hydroxy-azepane-1-carboxylic acid benzyl ester

To a solution of the epoxide from Example 1c (2.0 g, 8.1 mmol) in methanol:water (8:1 solution) was added NHCl (1.29 g, 24.3 mmol) and sodium azide (1.58 g, 24.30 mmol). The reaction was heated to 65-75° C. until complete consumption of the starting epoxide was observed by TLC analysis. The majority of the solvent was removed in vacuo and the remaining solution was partitioned between ethyl acetate and pH 4 buffer. The organic layer was washed with sat. NaHCO₃, water, brine dried (MgSO₄), filtered and concentrated. Column chromatography (20% ethyl acetate:hexanes) of the residue provided 1.3 g of the title compound: MS(EI) 291 (M+H⁺) plus 0.14 g of trans-4-hydroxy-3-azido-hexahydro-1H-azepine

e.) 4-Amino-3-hydroxy-azepane-1-carboxylic acid benzyl ester

To a solution of the azido alcohol of Example 1d (1.1 g, 3.79 mmol) in methanol was added triethyamine (1.5 mL, 11.37 mmol) and 1,3-propanedithiol (1.1 mL, 11.37 mmoL). The reaction was stirred until complete consumption of the starting material was observed by TLC analysis whereupon the reaction was concentrated in vacuo. Column chromatography of the residue (20% methanol:dichloromethane) provided 0.72 g of the title compound: MS(EI) 265 (M+H⁺).

f.) 4-((S)-2-tert-Butoxycarbonylamino-3-phenyl-propanoylamino)-3-hydroxy-azepan-1-carboxylic acid benzyl ester

To a solution of the amino alcohol of Example 1e (0.40 g, 1.3 mmol) in CH₂Cl₂ (13 mL) was added Boc-phenylalanine (0.35 g, 1.3 mmol), EDC (0.28 g, 1.5 mmol), HOBT (0.20 g, 1.5 mmol) and TEA (0.76 mL, 5.5 mmol). The reaction mixture was stirred under argon at room temperature for 20 hours. The reaction was diluted with ethyl acetate and washed successively with saturated K₂CO₃ and brine. The combined aqueous layers were then back extracted with ethyl acetate. The combined organic layers were dried over MgSO₄, filtered, and concentrated. Column chromatography (4% CH₃OH:CH₂Cl₂) yielded 0.58 g (86%) of as a white powder.

g.) [(S)-1-(3-Hydroxy-azepan-4-ylcarbamoyl)-2-phenyl-ethyl]-carbamic acid tert butyl ester

Palladium on carbon (0.095 g) was added to a solution of the compound from example 1f (0.58 g, 1.1 mmol) in methanol (11 mL). The reaction was stirred under a hydrogen atmosphere for 20 h. The reaction mixture was filtered through a celite plug and concentrated to provide 0.46 g of crude product which was of sufficient purity to carry to the next step with no further purification.

h.) [(S)-1-[3-Hydroxy-1-(pyridine-2-sulfonyl)-azepan-4-ylcarbamoyl]-2-phenyl-ethyl}-carbamic acid tert-butyl ester

To a vigorously stirred solution of the compound from Example 1 g (0.43 g, 1.1 mmol) in CH₂Cl₂ (8.0 mL) was added saturated NaHCO₃ (19 mL). Pyridinesulfonyl chloride (0.49 g, 2.8 mmol) was added and the resulting pale yellow reaction stirred at room temperature for 2 h. The reaction was diluted with CH₂Cl₂, layers separated, and the aqueous layer extracted with CH₂Cl₂. The combined organic layers were washed with brine, dried over Na₂SO₄, filtered, and concentrated. Column chromatography (5% CH₃OH:CH₂Cl₂) yielded 0.45 g (77%) of the title compound as a white powder.

i.) (S)-2-Amino-N-[3-hydroxy-1-(pyridine-2-sulfonyl)-azepan-4-yl]-3-phenyl-proprionamide

HCl (5.8 mL, 4.0 M in dioxane) was added to a solution of the compound of Example 1h (1.2 g, 2.3 mmol) in methanol (23 mL). The reaction was stirred at room temperature for 20 h. The reaction mixture was then concentrated in vacuo and azeotroped four times with toluene. The crude product was carried to the next step: MS(ESI) 419.4 (M+H)⁺.

j.) Quinoline-6-carboxylic acid {(S)-1-[3-hydroxy-1-(pyridine-2-sulfonyl)-azepan-4-ylcarbamoyl]-2-phenyl-ethyl} amide

To a mixture of the compound of Example 1i (0.10 g, 0.22 mmol), quinoline-6-carboxylic acid (0.038 g, 0.22 mmol), EDC (0.047 g, 0.25 mmol), and HOBT (0.033 g, 0.24 mmol) were added CH₂Cl₂ (2.1 mL) and TEA (0.12 mL, 0.86 mmol). The reaction mixture was stirred under argon at room temperature for 22 hours. The reaction was diluted with ethyl acetate and washed successively with saturated K₂CO₃ and brine. The combined aqueous layers were then back extracted with ethyl acetate. The combined organic layers were dried over MgSO₄, filtered, and concentrated. Column chromatography (4% CH₃OH:CH₂Cl₂) yielded 0.091 g (72%) of the title compound as a white powder.

k.) Quinoline-6-carboxylic acid {(S)-1-[3-oxo-1-(pyridine-2-sulfonyl)-azepan-4-ylcarbamoyl]-2-phenyl-ethyl}amide

To a solution of the alcohol of example 1j (0.089 g, 0.16 mmol) in CH₂Cl₂ (2.0 mL) was added Dess-Martin periodinane (0.11 g, 0.25 mmol). The reaction was stirred under argon at room temperature for 1.5 h whereupon it was diluted with CH₂Cl₂ and washed with 10% aqueous Na₂S₂O₃, two portions of saturated NaHCO₃, dried (Na₂SO₄), filtered, and concentrated. Column chromatography (1:2 hexanes:ethyl acetate) followed by preparative HPLC (40:60 hexanes:ethanol, 10μ 100A (R,R) Whelk-O, 25 cm×21.1 mm ID) provided 0.019 g of the faster eluting diastereomer and 0.019 g of the slower eluting diastereomer (combined 43% yield), both as white powders: ¹H NMR (400 MHz, CDCl₃) as a mixture of diastereomers δ 9.01 (d, 1H), 8.71 (m, 1H), 8.26 (m, 2H), 8.16 (d, 1H), 8.08 (d,1H), 7.96 (m, 2H), 7.49 (m, 2H), MS(ESI) 622.0 (M+H)⁺.

### Example 2

Preparation of Quinoline-6-carboxylic acid {(S)-2-naphthylen-2-yl-1-[3-oxo-1-(pyridine-2-sulfonyl)-azepan-4-ylcarbamoyl)-ethyl]-amide

Following the procedures example 1 f-k except substituting N-Boc-β-naphthylalanine for N-Boc-phenylalanine the title compound was prepared: ¹H NMR (400 MHz, CDCl₃) δ 9.00 (d, 1H), 8.68 (d, 1H), 8.26 (d, 1H), 8.17 (m, 1H), 8.07 (d, 1H), 7.97 (m, 2H), 7.85 (m, 3H), 7.74 (s, 1H), 7.48 (m, 6H), 7.21 (d, 1H), 6.69 (d, 1H), 5.07 (m, 2H), 4.56 (d, 1H), 4.09 (d, 1H), 3.68 (d, 1H), 3.51 (dd, 1H), 3.32 (dd, 1H), 2.63 (m, 1H), 2.18 (m, 2H), 1.78 (m, 1H), 1.40-1.26 (m, 1H); MS(ESI) 622.0 (M+H)⁺.

The above specification and Examples fully disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

We claim:

1. A method of inhibiting cathepsin L, comprising administering to a patient in need thereof an effective amount of a compound of Formula I: 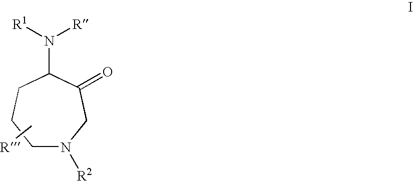
wherein:
R¹ is 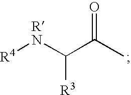
R² is H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹(R¹¹)NSO₂— 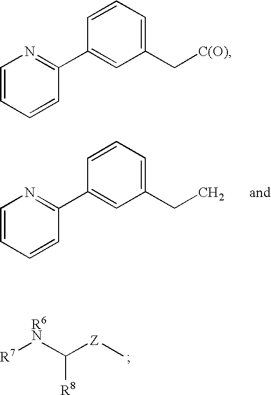
R³ is H, C₁₋₆alkyl, C₂₋₆alkenyl, C₂₋₆alkynyl, HetC₀₋₆alkyl and ArC₀₋₆alkyl;
R³ and R′ may be connected to form a pyrrolidine, piperidine or morpholine ring;
R⁴ is R⁵OC(O)—;
R⁵ is quinolin-6-yl;
R⁶ is H, C₁₋₆alkyl, Ar—C₀₋₆alkyl, or Het-C₀₋₆alkyl;
R⁷ is H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹⁴NC(O)—, or R¹⁰R¹⁴NC(S)—;
R⁸ is H, C₁₋₆alkyl, C₂₋₆alkenyl, C₂₋₆alkynyl, HetC₀₋₆alkyl or ArC₀₋₆alkyl;
R⁹ is C₁₋₆alkyl, C₃₋₆cycloalkyl-C₀₋₆alkyl, Ar—C₀₋₆alkyl or Het-C₀₋₆alkyl;
R¹⁰ is C₁₋₆alkyl, C₃₋₆cycloalkyl-C₀₋₆alkyl, Ar—C₀₋₆alkyl or Het-C₀₋₆alkyl;
R¹¹ is H, C₁₋₆alkyl, Ar—C₀₋₆alkyl, or Het-C₀₋₆alkyl;
R¹² is H, C₁₋₆alkyl, Ar—C₀₋₆alkyl, or Het-C₀₋₆alkyl;
R¹³ is H, C₁₋₆alkyl, Ar—C₀₋₆alkyl, or Het-C₀₋₆alkyl;
R¹⁴ is H, C₁₋₆alkyl, Ar—C₀₋₆alkyl, or Het-C₀₋₆alkyl;
R′ is H, C₁₋₆alkyl, Ar—C₀₋₆alkyl, or Het-C₀₋₆alkyl;
R″ is H, C₁₋₆alkyl, Ar—C₀₋₆alkyl, or Het-C₀₋₆alkyl;
R′″ is H, C₁₋₆alkyl, C₃₋₆cycloalkyl-C₀₋₆alkyl, Ar—C₀₋₆alkyl, or Het-C₀₋₆alkyl;
X is CH₂, S, or O; and
Z is C(O) or CH₂;
and pharmaceutically acceptable salts, hydrates and solvates thereof.

2. A method according to claim 1 wherein in said compound R³ is C₁₋₆alkyl and Ar—C₀₋₆alkyl.

3. A method according to claim 2 wherein in said compound R³ is isobutyl, napthalen-2-ylmethyl, benzyl, or benzyloxymethyl.

4. A method according to claim 1 wherein in said compound R′ is H.

5. A method according to claim 1 wherein in said compound R″ is H.

6. A method according to claim 1 wherein in said compound R′″ is H.

7. A method according to claim 1 wherein in said compound R″ and R′″ are both H.

8. A method according to claim 1 wherein in said compound:
R² is H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹R¹¹NSO₂—, 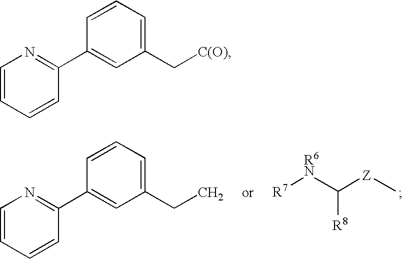
R⁶ is H, C₁₋₆alkyl, Ar—C₀₋₆alkyl, or Het-C₀₋₆alkyl;
R⁷ is H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹⁴NC(O)—, or R¹⁰R¹⁴NC(S);
R⁸ is H, C₁₋₆alkyl, C₂₋₆alkenyl, C₂₋₆alkynyl, HetC₀₋₆alkyl or ArC₀₋₆alkyl;
R⁹ is C₁₋₆alkyl, C₃₋₆cycloalkyl-C₀₋₆alkyl, Ar—C₀₋₆alkyl, or Het-C₀₋₆alkyl;
R¹⁰ is C₁₋₆alkyl, C₃₋₆cycloalkyl-C₀₋₆alkyl, Ar—C₀₋₆alkyl or Het-C₀₋₆alkyl; and
Z is C(O) or CH₂.

9. A method according to claim 8 wherein in said compound R² is R⁹SO₂.

10. A method according to claim 9 wherein in said compound R⁹ is Het-C₀₋₆alkyl.

11. A method according to claim 10 wherein in said compound R⁹ is pyridinyl or 1-oxy-pyridinyl.

12. A method according to claim 11 wherein in said compound R⁹ is pyridin-2-yl or 1-oxy-pyridin-2-yl

13. A method according to claim 12 wherein said compound is:
quinoline-6-carboxylic acid {(S)-naphthylen-2-yl-1-[(S)-oxo-1-(pyridine-2-sulfonyl)-azepan-4-yl carbamoyl]-ethyl}-amide, or
quinoline-6-carboxylic acid {(S)-1-[(S)-3-oxo-1-(pyridine-2-sulfonyl)-azepan-4-yl carbamoyl]-2-phenyl-ethyl}-amide; or
a pharmaceutically acceptable salt, hydrate or solvate thereof.

14. A method of treating a disease characterized by positive selection of CD4⁺T-cells by cortical thymic epithelial cells comprising inhibiting said positive selection of CD4⁺T-cells by cortical thymic epithelial cells by administering to a patient in need thereof an effective amount of a compound according to claim 1.
